(19) United States
(12) Patent Application Publication
(10) Pub. No.: US 2016/0346283 A1
(43) Pub. Date: Dec. 1, 2016

(54) Various Compounds as Autophagy Stimulants

(71) Applicant: Velgene 3 Limited, Szeged (HU)

(72) Inventors: Attila Erdos, Szeged (HU); Tibor Vellai, Szeged (HU)

(21) Appl. No.: 15/167,350

(22) Filed: May 27, 2016

Related U.S. Application Data
(63) Continuation of application No. PCT/EP2014/076163, filed on Dec. 1, 2014.

(30) Foreign Application Priority Data
Nov. 29, 2013 (GB) 1321126.3

(51) Int. Cl. A61K 31/517; A61K 31/429; A61K 31/4184

(52) U.S. Cl. CPC A61K 31/517; A61K 31/4184; A61K 31/429

(57) ABSTRACT

The invention relates to promoting autophagy in the treatment or prevention of autophagy related disorders, such as various forms of cancer; liver disease, myopathies of various origin; cardiovascular disorders, neurodegenerative disorders by using the compounds of the invention or their pharmaceutically acceptable salts.

### RELATED APPLICATIONS

This application is a continuation of International Application No. PCT/EP2014/076136, which designated the United States and was filed on Dec. 1, 2014, published in English.

This application claims priority under 35 U.S.C. §119 or 365 to Great Britain, Application No. 1321126.3, filed Nov. 29, 2013.

The entire teachings of the above application(s) are incorporated herein by reference.

The invention relates to promoting autophagy in the treatment or prevention of autophagy related disorders, such as various forms of cancer; liver disease, myopathies of various origin; cardiovascular disorders, neurodegenerative disorders by using the compounds of the invention or their pharmaceutically acceptable salts.

The invention also relates to the salts of the above molecules in any formulations such as but not limited to tablets, capsules, solutions and ointments. The invention further relates to suitable pharmaceutical compositions, which contain the above molecules as a combined preparation for simultaneous, separate or sequential use for the treatment and prevention of autophagy related diseases.

In addition, the above molecules can be used to extend the capabilities of humans or animals of leading a longer life called longevity.

### SUMMARY

It has been recently discovered that autophagy plays a crucial role in the homeostasis of the cell to maintain its normal function. It has been demonstrated that impaired or attenuated autophagic activity can lead to cancer, liver disease, various myopathies and neurodegenerative disorders and the deterioration of its normal level might also be responsible for shortening the life span. In parallel, it has also been demonstrated that stimulation of autophagy can lead to longevity.

Although the regulation of autophagy is a complex phenomenon, certain myotubularin proteins play a key role. It has been shown that MTMR14 and MTMR6—two proteins of this kind—have a central role by blocking autophagy by antagonizing the type III phosphatidylinositol 3-kinase VPS34 (stand for vacuolar protein sorting protein 34). This central mechanism of the pathway is conserved across the various species.

The inventors demonstrated that effective molecules dose dependently inhibit MTMR14 and or MTMR6 and subsequently significantly increases the autophagic activity of the cell, therefore it is effective in treating autophagy-dependent disorders. The inventors also demonstrated that by inhibiting MTMR14 or MTMR6 the life span significantly increased in various animal models, thus the effective molecules exert longevity attributes.

### BACKGROUND OF THE INVENTION

Autophagy is a highly regulated self-degradation process of eukaryotic cells. During autophagy, parts of the cytoplasm are sequestered by a double-membrane structure, thereby forming a vesicle-like structure called autophagosome. Autophagosome then fuses with a lysosome, and in the resulting structure called autolysosome the sequestered cargo becomes degraded by lysosomal hydrolases (proteases, nucleases, lipases and glicosylases). The end products of autophagic breakdown can be served as building blocks for the synthetic processes or provide energy for the cell under starvation. Thus, autophagy plays an essential role in the renewal of cellular components (macromolecule and organelle turnover) and primarily functions as a cell-protecting mechanism. Autophagic degradation is important in cell growth and proliferation, survival of cells, and in defense against intracellular microorganisms; in humans, diverse age-related pathological conditions such as cancer, neurodegenerative diseases (e.g., Alzheimer, Parkinson and Huntington disease), stroke, sarcopenia, immune deficiency and heart attack involve dysregulated autophagy.

A basic biochemical reaction that mediates the formation of the autophagic (isolation) membrane is catalyzed by a conserved kinase, PI3K-III (type III phosphatidylinositol 3-kinase). This enzyme converts phosphatidyl-inositol-3 phosphate into phosphatidyl-inositol-3,5 bisphosphate. Thus, PI3K-III is a critical component of the autophagic process. The molecular antagonists of PI3K-III involve certain myotubularin-related (MTMT) phosphatases. These MTMT enzymes can inhibit autophagic degradation. In genetic model systems and cell cultures, inhibition of mtm genes leads to a potent autophagy activation. Loss-of-function mutations in mtm genes can significantly extend lifespan, suppress neuronal cell death, and prevent muscle and other tissues from undergoing atrophy. A myotubularin protein (MTMT14) is implicated in fine tuning of autophagy.

We have aimed to develop specific MTMR14 inhibitors with the potential to stimulate the autophagic process.

### The Role of Autophagy in Physiology and Pathology

During autophagy, parts of the cytoplasm are sequestered into a double-membrane bound structure called an autophagosome, and then delivered into the lysosome lumen for enzymatic degradation. The resulting products of autophagic degradation are later utilized in anabolic processes or as cellular energy. Autophagy is basically responsible for the elimination of damaged or worn-out cellular components (dysfunctional and abnormal macromolecules and organelles). Autophagy also plays a key role in cellular stress response during starvation, in the regulation of cell growth, division and loss, in aging control and in the defense against intracellular pathogens. Defects in autophagy can lead to the development of various types of tumours, premature aging, various neurodegenerative disorders, muscle atrophy (sarcopenia), stroke, heart failure and infections caused by parasitic bacteria or viruses (Levine and Kroemer, 2008; Mizushima et al., 2008). Understanding the mechanisms and regulation of autophagy is therefore of utmost importance for biomedical, social and economic reasons. The most common fatal diseases of mankind normally develop at advanced ages. While the role of pathological functioning of several proteins (such as oncoproteins, tumour suppressors or aggregation-prone proteins) in the development of these diseases has been revealed over the past few decades, understanding the molecular and cytological bases of these processes remains in the forefront of current biological research. Therefore, it is clear that the pathological mechanisms underlying cancer, neurodegeneration and muscle atrophy—all of which are complex, multifactorial processes—are yet to be discovered. Interestingly, these diseases with apparently diverse origin, molecular basis and clinical picture have something else in common apart from the fact that they predominantly develop at advanced ages, and it is that they are all caused by damaged cellular components. Such types of molecular damage include dysfunctional, oxidized, misfolded, crosslinked or aggregated macromolecules. For example, oxidation of DNA may lead to single- or double-stranded breaks, and during the repair of these breaks the nucleotide sequence can change. The resulting mutations can trigger uncontrolled cell division. Protein aggregation can also lead to various neurodegenerative processes. Alzheimer's disease, for instance, is caused by the accumulation of β-amyloid and tau proteins, while Parkinson's disease is accompanied with the aggregation of α-synuclein in dopaminergic neurons. It is the gradual age-related accumulation of molecular damage, which drives the aging process.

Normal metabolic processes result in a continuous generation and accumulation of cellular damage. Various enzymes and the mitochondrial respiratory chain all produce reactive oxygen species (ROS), such as oxygen anions, superoxide and hydroxyl radicals, peroxides, which can oxidize macromolecules. The removal of ROS is essential for the maintenance of cellular homeostasis. Malfunction and deterioration of cellular repair systems are likely to be responsible for aging as well as for the incidence of most age-related diseases. Due to this remarkable molecular convergence, one in the close future may be able to modify (slow down) the rate at which the cells and tissues age and to delay the incidence of numerous age-related degenerative processes. The removal of damaged cellular components primarily occurs through autophagy. During autophagy (a term which is composed of Greek words “auto”—for self—and “phagein”—for eating—and means cellular self-digestion) parts of the cytoplasm are delivered to lysosomes through a regulated process, in which they are degraded by lysosomal hydrolases. Dysfunctional autophagy has been linked to the development of various geriatric diseases (cancer, neurodegenerative disorders, tissue atrophy, heart failure, stroke and microbial infections). Cytological aspects of autophagy were determined many decades ago.

Despite its medical significance, the genetic and molecular bases (that is the regulation and mechanism) of this process were only discovered very recently. There is a quite straightforward explanation for this discrepancy. Autophagic vacuoles are micron-sized and so autophagy in the past century could only be examined by electron microscopy. This idiosyncrasy has made it impossible to use efficient genetic methods (genetic screens) to identify autophagy-specific genes. It is quite obvious why no one undertook the task of detecting autophagy-deficient mutant organisms using electron microscopy. The breakthrough came with the study of autophagy in single-celled yeast. Yeast contains a single autophagic vacuole (an organelle analogous to the lysosome), which can already be identified by light microscopy. This finding was followed by a series of genetic screens to identify yeast autophagy-related genes (ATG). Identification of metazoan orthologs of yeast autophagy genes have opened the way to the molecular and functional (genetic) analysis of autophagy in higher organisms.

During autophagy, cellular components are translocated into the lysosome through a regulated process. Based on the method of translocation, three main types of autophagy can be distinguished: microautophagy, chaperon-mediated autophagy (CMA) and macroautophagy.

During microautophagy the lysosomal membrane directly engulfs parts of the cytoplasm (invagination). CMA, which does not occur in plant cells, is responsible for the degradation of proteins containing a specific pentapeptide motif, KFERQ. These proteins are marked by molecular chaperones and are transported to the lysosomes though the LAMP-2a (type 2a lysosome-specific membrane protein) receptor. Interestingly, α-synuclein, whose aggregation results in the development of Parkinson's disease also contains the KFERQ motif. Qualitatively, macroautophagy is the most significant protein and organelle degradation mechanism. For simplicity, the term autophagy is henceforth used as a synonym for macroautophagy. During this process, a double membrane structure is formed inside the cytoplasm, sequestrating cellular components (macromolecules and organelles) from the rest of the cell. When the membrane growing is completed, the resulting structure is called autophagosome (FIG. 1). The mature autophagosome then fuses with a lysosome to form an autolysosome, in which the segregated cellular components are degraded into building blocks.

One of the most remarkable features of autophagy is that it is in a tight connection with numerous signal transduction systems, environmental (nutrients, temperature, oxygen) and cellular factors (mitogens, growth factors, ATP levels) (FIG. 2).

Recent results suggest that autophagy acts as a downstream effector process in the regulation of cell growth, proliferation and death. On the other hand, depending on the actual cellular milieu, autophagy is one of the most important means of cell survival. For example, the effect of genetic pathways regulating cell division (such as the Ras, insulin/IGF-1, TGF-β, JNK, G-protein mediated and TOR signal transduction systems) are mediated by the autophagic process. Signal transduction pathways regulating aging (e.g. insulin/IGF-1, TGF-beta, JNK, TOR and Ras/ERK signalling) also converge on the autophagy gene cascade. In addition, biomedically highly important proteins such as p53, FoxO, E2F (a component of the retinoblastoma complex), FoxA, Sirt1 (a sirtuin) regulate the activity of certain autophagy genes directly (i.e. they function as transcription factors of autophagy genes). Therefore, it is evident that autophagy plays a role in the processes of aging, cell division and death.

Autophagy genes are vital in Drosophila and in C. elegans under both normal and starvation-stress induced conditions. In C. elegans, reduced levels of insulin/IGF-1 (insulin-like growth factor 1), TOR signal transduction pathways, mitochondrial respiration or caloric restriction each increase lifespan. The increased lifespan of these animals is autophagy-dependent: inactivation of autophagy genes suppresses the extension of lifespan. Furthermore, it has been demonstrated in insects that the activity (expression) of autophagy genes gradually decreases as the animal ages (as part of the normal aging process) and that overexpression of the autophagy protein Atg8 in the nervous system increases lifespan by 50%. Autophagy genes hence form an “anti-aging” pathway, onto which the effects of the signal transduction systems regulating longevity converge (FIG. 2). Autophagy is, therefore, a central regulatory mechanism of animal aging.

### The Mechanisms of Autophagy

Based on their function, the ATG genes can be classified into four groups: 1, genes mediating induction (nucleation); 2, genes that mediate isolation membrane growing; 3, members of the Atg8 conjugation system; 4, genes involved in recyclization (FIG. 3). Induction of autophagy is regulated by an Atg1 kinase complex. This complex contains other proteins, including Atg13 and Atg17. Under normal conditions, Atg13 is phosphorylated by the kinase target of rapamycin (TOR); in this state the complex is not able to initiate autophagy. Under starvation, however, TOR becomes inactivated, resulting in the dephosphorylated state of Atg13. Under these circumstances the Atg1 complex promotes autophagosome formation.

After induction, another kinase complex, whose central component is VPS34 (vacuolar protein sorting-associated protein), a type III phosphatidylinositol-3 kinase, mediates the synthesis of the growing isolation membrane. In addition to VPS34, this complex also includes Atg6, Atg14 and Atg15 proteins, and participates in the synthesis of other, non-autophagosomal membranes.

The growing isolation membrane should be identified as an autophagosomal membrane. This can be achieved by covalent binding (conjugation) of Atg8, a ubiquitin-like protein, to the membrane. Initially, Atg8 is a cytosolic, soluble protein (Atg8-I). Upon induction, the last amino acid (a glycine) of Atg8 becomes cleaved off from the protein, leaving a free carboxyl terminus that can bind to a membrane component, phosphatidyl-etanolamine (PE). The PE-bound form of Atg8 is insoluble (Atg8-II). It binds to the forming autophagosomal membranes. In the conjugation process of Atg8, numerous Atg proteins participate, including Atg3, 4, 5, 7, 12 and 16.

After autophagosome formation, its outer membrane fuses with a lysosome, generating thereby a structure called autolysosome, where the cargo (sequestered cytoplasmic materials) is degraded by acidic hydrolases. After autolysosome formation, several components of the autophagoc structure can be regained through recyclization.

### Myotubularin Phosphatases

The catalyst of the initial biochemical process during autophagy is a lipid kinase, PI3K-III, which phosphorylates phosphatidylinositol 3-phosphate (PtdIns3P) to phosphatidylinositol 3,5-bisphosphate (PtdIns3,5P)., which is essential for membrane formation. Thus, PI3K-III activity stimulates the formation of autophagosomes. The chemical process catalyzed by PI3K-III is an equilibrated biochemical reaction: myotubularin-related (MTMT) phosphatases

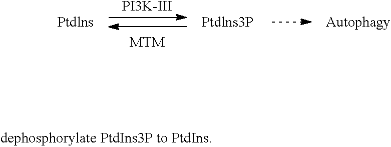

MTMT activity, therefore, results in the suppression of autophagy. This suggests that inhibition of MTMT activity can lead, in theory, to stimulation of autophagy. Indeed, it has been demonstrated that in C. elegans the suppression of certain mtm genes activates autophagy (to salvage the larval mortality of PI3K-III-mutant animals).

MTMR proteins form a conserved family of phosphatases. The human genome encodes 13 MTMR proteins (Robinson and Dixon, 2006). These paralogs differ in their structure and only certain types are suitable for efficiently dephosphorylating PtdIns3P. The lack of certain MTMR proteins during ontogeny might lead to the development of mendelian inherited diseases (e.g. myopathy, neuropathy or Charcot-Marie-Tooth syndrome). Out of the 13 human MTMR proteins only MTMR1 (myotubular myophaty), MTMR2 (type 4B1 Charcot-Marie-Tooth syndrome) and MTMR5/13 (infertility in mice) have so far been linked to pathological processes. It is worth to mention that the lack of MTMR proteins in adulthood (that is, after ontogeny) has not yet been linked to known human disease. This is very important from our point of view and for the concept of the present application: the specific-suppression of MTMR proteins does not result in degenerative disorders.

### DESCRIPTION OF THE INVENTION

In one aspect the present invention relates to the use of autophagy inducing compounds of Formula I, II and III or their pharmaceutically acceptable salts, tautomers, and hydrates thereof in promoting autophagy. The invention also relates to methods of promoting autophagy comprising administering to a subject a therapeutically effective amount of a compound of Formula I, II or III or their pharmaceutically acceptable salts, tautomers, and hydrates thereof

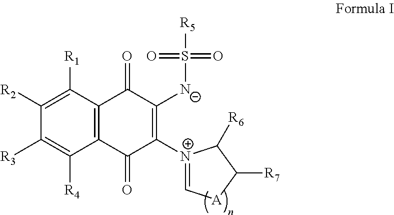

Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;

A is selected from C(R^{a})ₘ or NW wherein are each R^{a} is independently selected from hydrogen, branched or linear aliphatic, alicyclic, aryl, heterocyclic, heteroaryl, halo-, nitro-, hydroxyl, amino, and alkoxy;

n is 1 or 2;
m is 1 or 2; when m is 1 the bond between the carbon atoms attached to R⁶ and R⁷ is a single bond;

R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryl or heteroaryl ring

Preferably R¹, R², R³, and R⁴ are each independently selected from H, C₁₋₆ alkyl or halo-. Preferably at least one of R¹, R², R³, and R⁴ is H. More preferably at least two or at least three of R¹, R², R³, and R⁴ are H. Most preferably R¹, R², R³, and R⁴ are all H.

In a preferred embodiment R⁵ is an optionally substituted aryl group. Preferably the aryl group is substituted with a nitro group. In a preferred embodiment R⁵ is a nitrosubstituted phenyl group, preferably:

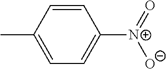

### Preferably n is 1. Preferably n is 1 and A is NR^{a}.

Preferably R^{a} are each independently selected from H, C₁₋₆ alkyl or halo-. More preferably R^{a} is H.

In one preferred embodiment R⁶ and R⁷ together form an optionally substituted 5 or 6 membered carbocyclic or heterocyclic ring. Preferably R⁶ and R⁷ together form an optionally substituted 6 membered heterocyclic ring. In a preferred embodiment the ring formed by R⁶ and R⁷ together with the group containing A is an optionally substituted heteroaryl group, preferably a benzimidazoyl group. In a preferred embodiment the group containing A is:

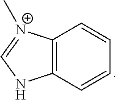

In a preferred embodiment the compound of Formula I is T0501-7132:

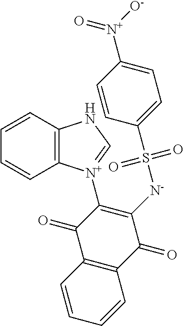 [SMILES: O=C1C([N-]S(=O)(=O)c2ccc([N+](=O)[O-])cc2)=C([n+]2c[nH]c3ccccc32)C(=O)c2ccccc21]

In a further aspect the invention relates to compounds of Formula II:

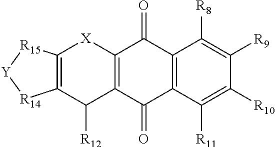

Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;

R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;

R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;

X is a heteroatom selected from sulphur, oxygen or N—R¹⁸, where R¹⁸ is selected from hydrogen, branched or linear aliphatic, alicyclic, aryl, heterocyclic, heteroaryl halo-, nitro-, hydroxyl, amino, and alkoxy;

Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy;

Preferably R⁸, R⁹, R¹⁰, and R¹¹ are each independently selected from H, C₁₋₆ alkyl or halo-. Preferably at least one of R⁸, R⁹, R¹⁰, and R¹¹ is H. More preferably at least two or at least three of R⁸, R⁹, R¹⁰, and R¹¹ are H. Most preferably R⁸, R⁹, R¹⁰, and R¹¹ are all H.

In a preferred embodiment R¹² is an optionally substituted aryl group. Preferably the aryl group is substituted with a hydroxy group. In a preferred embodiment R¹² is a hydroxysubstituted phenyl group, preferably:

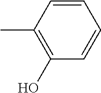

### Preferably Y is C(O).

In a preferred embodiment X is S.
Preferably R¹⁴ is a heteroatom, more preferably S.
R¹⁵ is preferably a heteroatom, more preferably N—R¹⁷. Most preferably R¹⁵ is NH.

Preferably the compound of Formula II is T544-1567:

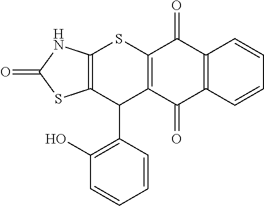 [SMILES: O=C1C2=C(C(=O)c3ccccc31)C(c1ccccc1O)c1sc(=O)[nH]c1S2]

In a further aspect the invention relates to compounds of Formula III:

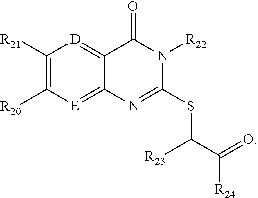

### Wherein

R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;

R²² is selected from hydrogen, branched or linear aliphatic, alicyclic, aryl, heterocyclic, heteroaryl, halo-, nitro-, hydroxyl, amino, cyano, and alkoxy;

R²³ selected from hydrogen, branched or linear aliphatic, alicyclic, aryl, heterocyclic, heteroaryl, halo-, nitro-, hydroxyl, amino, and alkoxy group;

R²⁴ is selected from alicyclic, aryl, heteroaryl or heterocyclic group;

D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy. Preferably R²⁰ and R²¹ are each independently selected from H, C₁₋₆ alkyl or halo-. Preferably at least one of R²⁰ and R²¹ is H. More preferably both R²⁰ and R²¹ are H.

In a preferred embodiment R²² is selected from hydrogen, branched or linear aliphatic, alicyclic, aryl, heteroaryl and heterocyclic groups. More preferably R²² is a alicyclic, more preferably an unsubstituted alicyclic. In a preferred embodiment R²² is cyclopentane.

Preferably R²⁴ is an optionally substituted aryl or heteroaryl group. Preferably R²⁴ is a heteroaryl, more preferably indole. In a preferred embodiment is an unsubstituted indole group, preferably:

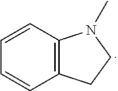

Preferably R²⁵ is selected from H, C₁₋₆ alkyl or halo-. Preferably R²⁵ is H.
Preferably D or E is CH₂. More preferably D and E are CH₂.
Preferably the compound of formula III is T558-0696:

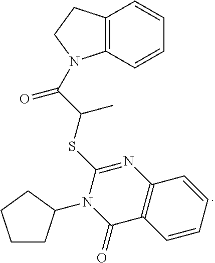 [SMILES: CC(Sc1nc2ccccc2c(=O)n1C1CCCC1)C(=O)N1CCc2ccccc21]

In a further aspect the invention relates to a compound of Formula I, II or III for use in a method of promoting autophagy.

In one aspect the invention relates to a method of promoting autophagy, comprising administering to the subject a therapeutically effective amount of autophagy inducing compounds of Formula I, II or III or their pharmaceutically acceptable salt, prodrug or tautomer thereof.

In another aspect the the invention relates to a method of treating an autophagy related disorder, comprising administering to the subject a therapeutically effective amount of autophagy inducing compounds of Formula I, II or III or their pharmaceutically acceptable salt, prodrug or tautomer thereof.

In an additional aspect the invention relates to a compound of Formula I, II or III for use in a method of treating an autophagy related disorder.

In another aspect the the invention relates to a method of promoting longevity, comprising administering to the subject a therapeutically effective amount of autophagy inducing compounds of Formula I, II or III or their pharmaceutically acceptable salt, prodrug or tautomer thereof.

In a further aspect the invention relates to a compound of Formula I, II or III for use in a method of promoting longevity.

In one aspect the the invention relates to a method of alleviating or preventing premature ageing, comprising administering to the subject a therapeutically effective amount of autophagy inducing compounds of Formula I, II or III or their pharmaceutically acceptable salt, prodrug or tautomer thereof.

In a further aspect the invention relates to a compound of Formula I, II or III for use in a method of alleviating or preventing premature ageing.

In another aspect the the invention relates to a method of alleviating or preventing premature ageing, comprising administering to the subject a therapeutically effective amount of autophagy inducing compounds of Formula I, II or III or their pharmaceutically acceptable salt, prodrug or tautomer thereof.

In a further aspect the invention relates to a compound of Formula I, II or III for use in a method of alleviating or preventing premature ageing.

### DEFINITIONS

For the purpose of the present invention, an aliphatic group is a hydrocarbon moiety that may be straight chain or branched and may be completely saturated, or contain one or more units of unsaturation, but which is not aromatic. The term “unsaturated” means a moiety that has one or more double and/or triple bonds. The term “aliphatic” is therefore intended to encompass alkyl, alkenyl or alkynyl groups, and combinations thereof. An aliphatic group is preferably a C₁₋₂₀ aliphatic group, that is an aliphatic group with 1, 2, 3, 4, 5, 6, 7, 8, 9, 10, 11, 12, 13, 14, 15, 16, 17, 18, 19 or 20 carbon atoms. Preferably, an aliphatic group is a C₁₋₁₅ aliphatic, more preferably a C₁₋₁₂ aliphatic, more preferably a C₁₋₁₀ aliphatic, even more preferably a C₁₋₈ aliphatic, such as a C₁₋₆ aliphatic group.

An alkyl group is preferably a “C₁₋₂₀ alkyl group”, that is an alkyl group that is a straight or branched chain with 1 to 20 carbons. The alkyl group therefore has 1, 2, 3, 4, 5, 6, 7, 8, 9, 10, 11, 12, 13, 14, 15, 16, 17, 18, 19 or 20 carbon atoms. Preferably, an alkyl group is a C₁₋₁₅alkyl, preferably a C₁₋₁₂alkyl, more preferably a C₁₋₁₀alkyl, even more preferably a C₁₋₈alkyl, even more preferably a C₁₋₆alkyl group. In certain embodiments, an alkyl group is a “C₁₋₆ alkyl group”, that is an alkyl group that is a straight or branched chain with 1 to 6 carbons. The alkyl group therefore has 1, 2, 3, 4, 5 or 6 carbon atoms. Specifically, examples of “C₁₋₂₀ alkyl group” include methyl group, ethyl group, n-propyl group, iso-propyl group, n-butyl group, iso-butyl group, sec-butyl group, tert-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, n-nonadecyl group, n-eicosyl group, 1,1-dimethylpropyl group, 1,2-dimethylpropyl group, 2,2-dimethylpropyl group, 1-ethylpropyl group, n-hexyl group, 1-ethyl-2-methylpropyl group, 1,1,2-trimethylpropyl group, 1-ethylbutyl group, 1-methylbutyl group, 2-methylbutyl group, 1,1-dimethylbutyl group, 1,2-dimethylbutyl group, 2,2-dimethylbutyl group, 1,3-dimethylbutyl group, 2,3-dimethylbutyl group, 2-ethylbutyl group, 2-methylpentyl group, 3-methylpentyl group and the like. Alkenyl and alkynyl groups are preferably “C₂₋₂₀alkenyl” and “C₂₋₂₀alkynyl”, more preferably “C₂₋₁₅alkenyl” and “C₂₋₁₅alkynyl”, even more preferably “C₂₋₁₂alkenyl” and “C₂₋₁₂alkynyl”, even more preferably “C₂₋₁₀alkenyl” and “C₂₋₁₀alkynyl”, even more preferably “C₂₋₈alkenyl” and “C₂₋₈alkynyl”, most preferably “C₂₋₆alkenyl” and “C₂₋₆alkynyl” groups respectively.

The term “non-branched”, used interchangebly with “simple”, as used herein refers to a straight chain alkyl group. Preferably, a simple alkyl group as referred to herein is a C₁₋₆ alkyl group.

An alicyclic group is a saturated or partially unsaturated cyclic aliphatic monocyclic or polycyclic (including fused, bridging and spiro-fused) ring system which has from 3 to 20 carbon atoms, that is an alicyclic group with 3, 4, 5, 6, 7, 8, 9, 10, 11, 12, 13, 14, 15, 16, 17, 18, 19 or 20 carbon atoms. Preferably, an alicyclic group has from 3 to 15, more preferably from 3 to 12, even more preferably from 3 to 10, even more preferably from 3 to 8 carbon atoms. The term “alicyclic” encompasses cycloalkyl, cycloalkenyl and cycloalkynyl groups. It will be appreciated that the alicyclic group may comprise an alicyclic ring bearing one or more linking or non-linking alkyl substitutents, such as —CH₂— cyclohexyl.

Cycloalkyl, cycloalkenyl and cycloalkynyl groups have from 3 to 20 carbon atoms. The cycloalkyl, cycloalkenyl and cycloalkynyl groups therefore have 3, 4, 5, 6, 7, 8, 9, 10, 11, 12, 13, 14, 15, 16, 17, 18, 19 or 20 carbon atoms. Cycloalkyl, cycloalkenyl and cycloalkynyl groups preferably have from 3 to 15, more preferably from 3 to 12, even more preferably from 3 to 10, even more preferably from 3 to 8 carbon atoms. When an alicyclic group has from 3 to 8 carbon atoms, this means that the alicyclic group has 3, 4, 5, 6, 7 or 8 carbon atoms. Specifically, examples of the C₃₋₂₀ cycloalkyl group include cyclopropyl, cyclobutyl, cyclopentyl, cyclohexyl, cycloheptyl, adamantyl and cyclooctyl.

The term “heterocyclyl” or “heterocyclic” refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, acridinyl, azepinyl, benzimidazolyl, benzindolyl, benzoisoxazolyl, benzisoxazinyl, benzodioxanyl, benzodioxolyl, benzofuranonyl, benzofuranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, [beta]-carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4-dithianyl, furanonyl, furanyl, imidazolidinyl, imidazolinyl, imidazolyl, imidazopyridinyl, imidazothiazolyl, indazolyl, indolinyl, indolizinyl, indolyl, isobenzotetrahydro furanyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthyridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4-piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydro furanyl, tetrahydroisoquinolinyl, tetrahydropyranyl, tetrahydrothienyl, tetrazolyl, thiadiazolopyrimidinyl, thiadiazolyl, thiamorpholinyl, thiazolidinyl, thiazolyl, thienyL triazinyl, triazolyl, and 1,3,5-trithianyl. In certain embodiments, heterocyclic may also be optionally substituted with one or more substituents as described herein.

An aryl group is a monocyclic or polycyclic ring system having from 5 to 20 carbon atoms. An aryl group is preferably a “C₆₋₁₂ aryl group” and is an aryl group constituted by 6, 7, 8, 9, 10, 11 or 12 carbon atoms and includes condensed ring groups such as monocyclic ring group, or bicyclic ring group and the like. Specifically, examples of “C₆₋₁₀ aryl group” include phenyl group, biphenyl group, indenyl group, naphthyl group or azulenyl group and the like. It should be noted that condensed rings such as indan and tetrahydro naphthalene are also included in the aryl group.

A heteroaryl group is an aryl group having, in addition to carbon atoms, from one to four ring heteroatoms which are preferably selected from O, S, and N. A heteroaryl group preferably has from 5 to 20, more preferably from 5 to 14 ring atoms. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to carbazolyl, benzindolyl, phenanthrollinyl, acridinyl, phenanthridinyl, and xanthenyl. In certain embodiments, heteroaryl may also be optionally substituted with one or more substituents as described herein.

An alkoxy group is preferably a “C₁₋₂₀ alkoxy group”, more preferably a “C₁₋₁₅ alkoxy group”, more preferably a “C₁₋₁₂ alkoxy group”, more preferably a “C₁₋₁₀ alkoxy group”, even more preferably a “C₁₋₈ alkoxy group”, even more preferably a “C₁₋₆ alkoxy group” and is an oxy group that is bonded to the previously defined C₁₋₂₀ alkyl, C₁₋₁₅ alkyl, C₁₋₁₂ alkyl, C₁₋₁₀ alkyl, C₁₋₈ alkyl, or C₁₋₆ alkyl group respectively. Specifically, examples of “C₁₋₂₀ alkoxy group” include methoxy group, ethoxy group, n-propoxy group, iso-propoxy group, n-butoxy group, iso-butoxy group, sec-butoxy group, tert-butoxy group, n-pentyloxy group, iso-pentyloxy group, sec-pentyloxy group, n-hexyloxy group, iso-hexyloxy group, n-hexyloxy group, n-heptyloxy group, n-octyloxy group, n-nonyloxy group, n-decyloxy group, n-undecyloxy group, n-dodecyloxy group, n-tridecyloxy group, n-tetradecyloxy group, n-pentadecyloxy group, n-hexadecyloxy group, n-heptadecyloxy group, n-octadecyloxy group, n-nonadecyloxy group, n-eicosyloxy group, 1,1-dimethylpropoxy group, 1,2-dimethylpropoxy group, 2,2-dimethylpropoxy group, 2-methylbutoxy group, 1-ethyl-2-methylpropoxy group, 1,1,2-trimethylpropoxy group, 1,1-dimethylbutoxy group, 1,2-dimethylbutoxy group, 2,2-dimethylbutoxy group, 2,3-dimethylbutoxy group, 1,3-dimethylbutoxy group, 2-ethylbutoxy group, 2-methylpentyloxy group, 3-methylpentyloxy group and the like.

An amino group is preferably —NH₂, —NHR³⁰ or —N(R³⁰)₂ wherein R³⁰ can be an aliphatic, alicyclic, aryl or heteroaryl group as defined above. It will be appreciated that when the amino group is N(R³⁰)₂, each R³⁰ group can be independently selected from an aliphatic, alicyclic, heteroalicyclic, heteroaryl or an aryl group as defined above. In certain embodiments, each R³⁰ is independently an unsubstituted aliphatic, alicyclic or aryl. Preferably R³⁰ is methyl, ethyl, or propyl.

The term “halide” or “halogen” are used interchangeably and, as used herein mean a fluorine atom, a chlorine atom, a bromine atom, an iodine atom and the like, preferably a fluorine atom, a bromine atom or a chlorine atom, and more preferably a fluorine atom or a bromine atom.

The term “nitro” group refers to N(O)H or NO₂.

An aliphatic, alkyl, alicyclic, heterocycle, cycloalkyl, aryl, or heteroaryl group as referred to in respect or any of the chemical moeities described herein, may be unsubstituted or may be substituted by one or more substituents independently selected from the group consisting of halo, aliphatic, —OR^{o}, —R^{o}, —SR^{o}, NHR^{o}, —NR^{o}₂, —COR^{o}, —COOR^{o}, —NH₂, —NO₂, —OH, —COOH, —CN, hydroxyalkyl, alkylcarbonyloxy, alkoxycarbonyl, alkylcarbonyl or alkylsulfonylamino, wherein R^{o} is an optionally substituted aliphatic (preferably alkyl), alicyclic (preferably aryl or cycloalkyl) or heterocycle (preferably heteroaryl or heterocycloalkyl) optionally substituted with or with any one or more of substituents independently selected from halo, aliphatic, —OR, —R, —SR, NHR, —NR₂, —COR, —COOOR, —NH₂, —NO₂, —OH, —COOH, —CN, hydroxyalkyl, alkylcarbonyloxy, alkoxycarbonyl, alkylcarbonyl or alkylsulfonylamino, wherein R is as defined for R^{o}, substituted or unsubstituted. Preferred substituents include halo, lower alkyl, alkylamino, —NH₂, NO₂, —OH, —CN, alkoxy or alkoxycarbonyl.

The terms “prodrug” mean a covalently-bonded derivative or carrier of the compound of the invention which undergoes at least some biotransformation prior to exhibiting its pharmacological effect(s). In general, such prodrugs have metabolically cleavable groups and are rapidly transformed in vivo to yield the compound of the invention, for example, by hydrolysis in blood, and generally include esters and amide analogs of the analogs. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the analogs using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987. In some embodiments, the compound may be a prodrug that, when administered to the subject becomes biologically active.

Compounds of the invention can be obtained from molecule banks such as ENAMINE (www.enamine.net).

Autophagy is the major catabolic process of eukaryotic cells that degrades and recycles damaged macromolecules and organelles. Promoting autophagy as used herein means increasing the autophagic activity within a cell or organism as compared to the rate of autophagy in the absence of treatment.

Preferably, the invention relates to autophagy inducing compounds of Formula I, II or III or their tautomers, prodrugs and pharmaceutically acceptable salt thereof for use in a method of treating an autophagy related disorder. As used herein an “autophagy related disorder” is selected from cancer, stroke, sarcopenia, infection, immune system deficiencies, liver disease, neurodegenerative diseases and cardiac disorders.

As used herein, a “neurodegenerative disease” is selected from, but not limited to Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjögren-Batten disease), Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), MELAS—Mitochondrial Encephalopathy, Lactic Acidosis and Stroke, Multiple System Atrophy, Multiple sclerosis, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Spinocerebellar ataxia (multiple types with varying characteristics), Spinal muscular atrophy, Steele-Richardson-Olszewski disease, Tabes dorsalis, Tay-Sachs Disease, and Toxic encephalopathy. Preferred neurodegenerative diseases include Alzheimer's disease.

The cancer can be selected from, but no limited to bone cancer, colon cancer, multiple myeloma, gastric cancer, colorectal cancer, prostate cancer, cervical cancer, lung cancer, pancreatic cancer, medulloblastoma, liver cancer, parathyroid cancer, endometrial cancer or breast cancer.

Liver disease includes, but is not limited to hepatitis, including viral hepatitis and autoimmune hepatitis; hepatitis steatosis; and cirrhosis.

Cardiac disorders include but are not limited to stroke, cardiac atrophy, heart attacks (myocardial infections), cardiomyopatry and transient ischemic attacks (TIAs). Preferably the cardiac disorder is a stroke or heart attack.

In a further aspect the present invention relates to autophagy inducing compounds or their pharmaceutically acceptable salt thereof for use in a method of promoting longevity or for alleviating or preventing premature ageing.

As used herein “promoting longevity” means increasing the expected life span of a patient. For humans the expected life span is 77-90 years in developed countries and 32-80 years in developing countries. The use of autophagy inducing compounds or their pharmaceutically acceptable salt thereof may increase the expected life span by 1-5%.

Promoting longevity also includes increasing the length of time a person can lead an active lifestyle without suffering from conditions associated with old age such as dementia, painful or reduced movement of limbs for example due to arthritis, or decreased cardiovascular function. The ‘active’ phase of a subject's life can be increased by 1-10 years, preferably 3-8 years, or 4-6 years.

“Premature ageing” as used herein refers to appearance of the signs of aging earlier than expected i.e. before old age. This includes early onset of conditions associated with old age such as degeneration of eyesight, dementia, impaired movement, cardiac conditions, as well as diseases such as Cockayne's syndrome. Premature ageing in the skin can be associated with the appearance of wrinkles, sun or liver spots, and thinning of the skin, at an earlier age than expected.

The compounds of the invention may be provided as the free compound or as a suitable salt or hydrate thereof. Salts should be those that are pharmaceutically acceptable and salts and hydrates can be prepared by conventional methods, such as contacting a compound of the invention with an acid or base whose counterpart ion does not interfere with the intended use of the compound. Examples of pharmaceutically acceptable salts include hydrohalogenates, inorganic acid salts, organic carboxylic acid salts, organic sulfonic acid salts, amino acid salt, quaternary ammonium salts, alkaline metal salts, alkaline earth metal salts and the like.

The compounds, salts and prodrugs of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds.

Compounds of the invention containing one or more asymmetric carbon atoms can exist as two or more stereoisomers. Where a compound of the invention contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

The compounds of the invention can be provided as a pharmaceutical composition. The pharmaceutical composition may additionally comprise a pharmaceutically acceptable excipient for example a pharmaceutically acceptable carrier and/or a pharmaceutically acceptable diluent. Suitable carriers and/or diluents are well known in the art and include pharmaceutical grade starch, mannitol, lactose, magnesium stearate, sodium saccharin, talcum, cellulose, glucose, sucrose (or other sugar), magnesium carbonate, gelatin oil, alcohol, detergents, emulsifiers or water (preferably sterile).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by the oral (including buccal or sublingual), rectal or topical (including buccal, sublingual or transdermal) route. Such compositions may be prepared by any method known in the art of pharmacy, for example by admixing the active ingredient with a carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions.

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurised aerosols, nebulizers or insufflators. Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts, buffers, coating agents or antioxidants. They may also contain an adjuvant and/or therapeutically active agents in addition to the substance of the present invention.

Dosages of the substance of the present invention can vary between wide limits, depending upon a variety of factors including the disease or disorder to be treated, the age, weight and condition of the individual to be treated, the route of administration etc. and a physician will ultimately determine appropriate dosages to be used.

The compositions comprising autophagy inducing compounds for use in the invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research.

Pharmaceutical formulations adapted for topical administration may be formulated as ointments, creams, suspensions, lotions, powders, solutions, pastes, gels, sprays, aerosols or oils.

For applications to the eye or other external tissues, for example the mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists which may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Preferred unit dosage formulations are those containing a daily dose or sub-dose, as herein above recited, or an appropriate fraction thereof, of an active ingredient.

It should be understood that in addition to the ingredients particularly mentioned above, the formulations may also include other agents conventional in the art having regard to the type of formulation in question, for example those suitable for oral administration may include flavouring agents.

Autophagy inducing compounds for use in the present invention may be administered in combination with one or more other active ingredients known to treat the disease of interest. Autophagy inducing compounds or their pharmaceutically acceptable salts thereof can be adapted for the simultaneous, separate or sequential use with one or more other active ingredients for the treatment and prevention of these diseases.

The invention will now be described with reference to the following non-limiting examples which refer to the Figures described below.

### DESCRIPTION OF FIGURES

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1a shows the basal activity of autophagy in HeLa cells used as a negative control (0.1% DMSO).

FIG. 1b shows the effect of 100 m Bafilomycin A1 on HeLa cells which inhibits the fusion process between autophagasomes and lysosomes.

FIG. 1c shows the results of using 200 nM rapamycin which is a known autophagy inducer, as a positive control.

FIG. 1d shows the results of treating HeLa cells with 10.0 μM T558-0696.

FIG. 1e shows the results of treating HeLa cells with 10.0 μM T0501-7132.

FIG. 1f compares the changes in the cytoplasmic GFP/GFP ratios induced by the autophagy inducing compounds.

FIG. 1g charts the summary of the dose dependent stimulating effects of different autophagy stimulating compounds.

FIG. 2a shows the results for the control non-treated, feeding wandering (90 hours) L3 larva (fat body).

FIG. 2b shows the results following treatment with 1.0 uM T558-0696 for feeding wandering (90 hours) L3 larva (fat body).

FIG. 2c shows the results of treatment with 10.0 uM T558-0696 in starved, wandering (90 hours) L3 larva (fat body).

FIG. 3a shows the effect of various doses of autohagy inducing compounds treatment on lifespan in Drosophilia.

FIGS. 3b and c shows the results of statistical calculations.

FIG. 4a shows the pancreas of a mouse from the control group treated with 100 uM DMSO.

FIG. 4b show autophagasomes and autolysosomes (indicated by arrows) present in the pancreas of mouse treated with 50 uMol T558-0696.

FIG. 4c show authophagasomes and autolysosomes (indicated by arrows) in the liver of mice treated with 50 uMol T558-0696.

### EXAMPLES

### 1) The Effects of Autophagy Inducing Compounds on Human Cell Line (HeLa) with Autophagy Reporting Proteins

### Methods

The GFP-RFP-LC3 HeLa cell line (Settembre et al, Science, 2011) was cultured in DMEM (Dulbecco's Modified Eagle's Medium, Sigma, D7777) containing 4500 mg/l glucose, 10% heat inactivated FCS (Merck), 40 μg/ml gentamycin (Hungaropharma) and 600 μg/ml G418 (Sigma, G8168).

Drug candidates T558-0696, T0501-7132 and T544-1567 were obtained from the ENAMINE molecule bank.

3×10⁴ cells were plated onto 13 mm diameter poly-D-lysine coated coverslips in 24-well plates (Greiner) with 24 hours before the treatment. Cells were exposed to 1 or 10 μM drug-candidate for 6 hours. As controls, 0.1 and 1% DMSO, 200 nM rapamycin (autophagy inductor) and 100 nM bafilomycin A1 (an autophagy inhibitor) were used.

For fluorescence microscopy, cells were fixed in 4% paraformaldehyde (Taab) and mounted in mowiol 4.88 (Polysciences) supplemented with bis-benzimide (Sigma) for nuclei staining. 5 epifluorescent pictures were taken in each condition by a BX51 microscope (Olympus) fitted with a FluoViewII camera and the AnalysisPro software (Olympus), using a 60×/1.4 oil Plan objective and the appropriate filter sets (DAPI: BP330-385/DM400/BA420; GFP: BP460-500/DM505/BP510-560; RFP: BP480-550/DM570/BA590).

RFP intensity shows both soluble LC3 molecules and activated LC3 along the whole autophagy process (late stages included). While GFP intensity shows soluble LC3 molecules and LC3 only in the early stages of autophagy, as GFP fluorescence is bleached by the acidic pH of the lysosome in late autophagosomes.

RFP: red fluorescence protein—autolysosome (mature autophagic compartment)
GFP: green fluorescence protein
Atg8: Autophagy-related protein 8—the most frequently used marker for autophagy
Yellow: RFP and GFP (merged)—autophagosome (the primary autophagic structure)

### Results

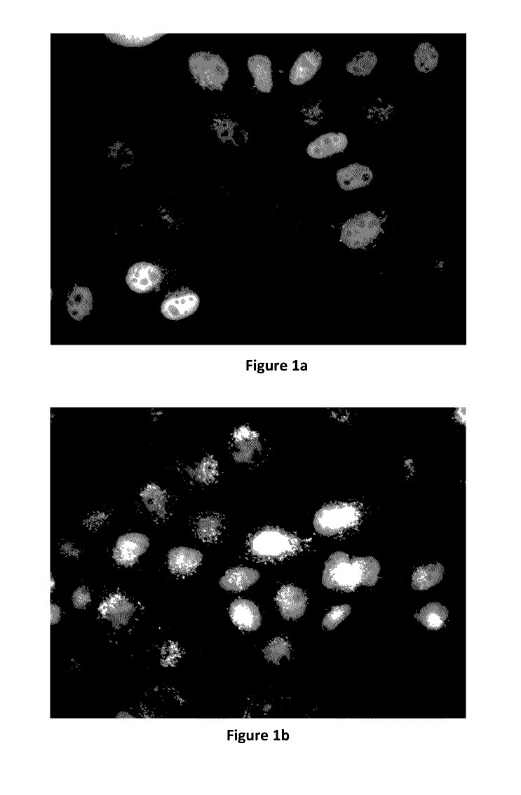

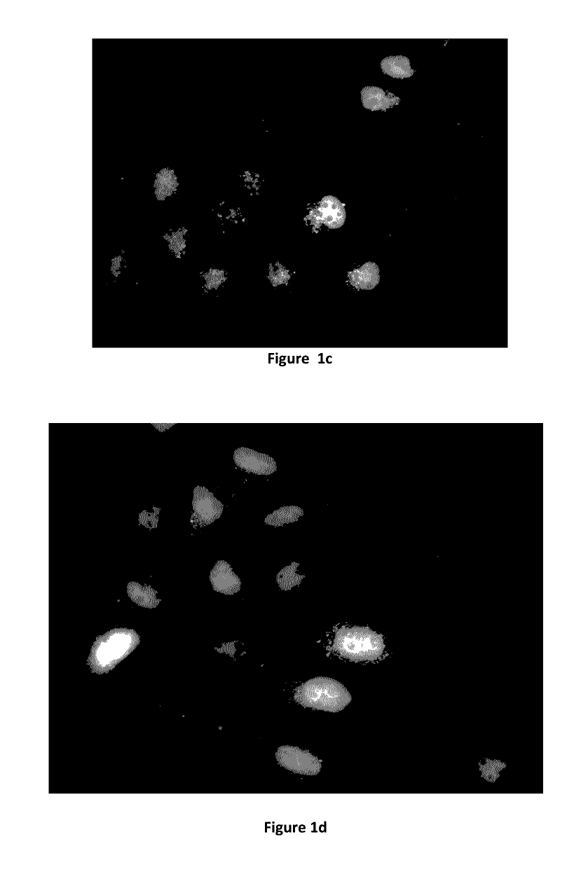

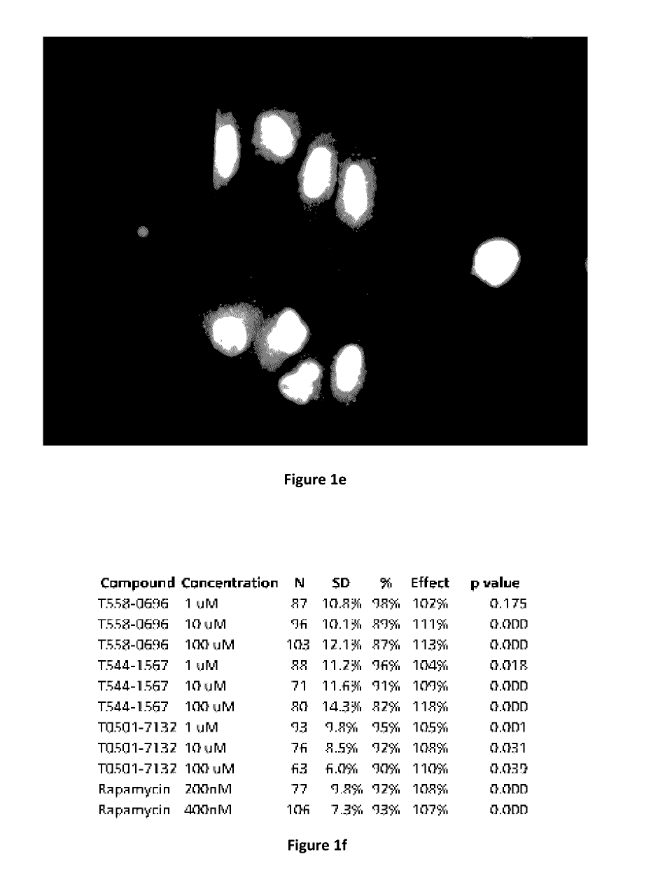

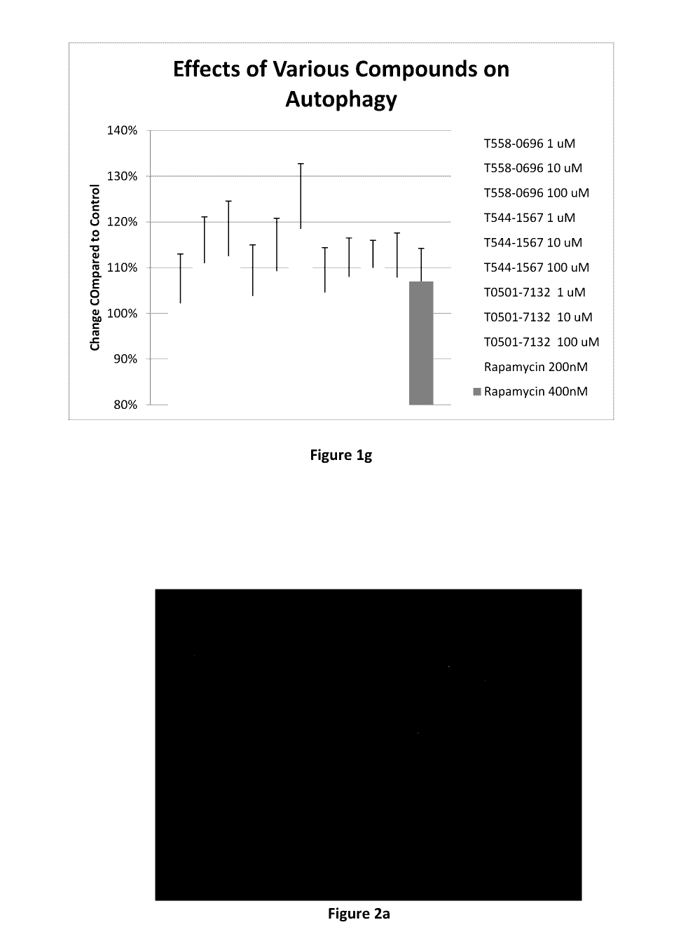

FIG. 1a shows the control (DMSO) culture, some background expression shows basal activity of autophagy in human HeLa cells. Yellow dots correspond to growing autophagosomes (before fusing with lysosomes); in these compartments bot RFP and GFP are active, resulting in yellow coloring. Red dots indicate autolysosomes (which result from the fusion of autophagosomes with lysosomes), whose lumen is acidic that leads to the degradation of GFP. Thus, autolysosomes are red.

Bafilomycin A1 inhibits the fusion process between autophagosomes and lysosomes (FIG. 1b). Thus, the autophagic process is blocked at an early stage upon treatment with this compound. Under this condition, all autophagic structures are represented by autophagosomes, causing thereby the appearance of yellow dots only (merged from red and green colors). As autphagy cannot proceed further yellow foci accumulate in large quantities.

Rapamycin is known as a potent autophagy inducer and was used as positive control (FIG. 1c). It actually inhibits the kinase target of rapamycin (TOR), which serves as an important upstream negative regulator of the autophagic process. Rapamycin treatment causes a huge accumulation of autolysosomes (red dots), but the presence of several autophagosomes (yellow dots) are also evident.

As it can be seen on FIG. 1d, 10.0 μM T558-0696 increases the number of red foci (autolysosomes), compared to negative control.

Similarly 10.0 μM of T0501-7132 also induces the accumulation the autolysosomes—red dots (FIG. 1e).

A larger dose (50 uM) of T0501-7132 shows markedly elevated activity. (FIG. 1f)

The numeric analysis of the samples using AnalysisPro software demonstrated markedly significant and dose dependent autophagy induction in certain concentrations. By calculating the change of the cytoplasmic RFP/GFP ratio induced by the administration of various doses of effective molecules demonstrates the effect. (FIG. 1g)

As it can be seen on the Summary chart (FIG. 1h), the autophagy inducing compounds show a dose dependent increase in autophagy comparable to the high dose of Rapamycin.

### 2) The Effects of Autophagy Inducing Compounds in Drosophila melanogaster Transgenic for an mCherry:Atg8 Reporter

### Methods

Flies with the genotype of hsFlp:pAct<CD2<Gal4, UAS-nlsGFP, r4:mCherry:Atg8a were used. Microscopic images were taken from the fat body of wandering larvae at the L3 stage. Note that in well-fed larvae at the same stage, no autophagic activity is visible. 2 hours before the test, 90-94 hours larvae were transferred onto yeast suspension (0.5 g yeast extract 2.5 ml water, homogenization and boiling). 1 g instant medium (Carolina, Formula 4-24 Instant Drosophila medium) and 4 ml H₂O were added to the suspension, Then, DMSO-solved 1 mM active sustances were added, supplemented to 6.5 ml of final volume (final concentration is 10-100 mM). Larvae were cultured in this medium for 2 hours. Preparation of fat bodies was performed in PBS solution. For covering, 8:2 ratio of glycerine:PBS was used (supplemented with 10 mM Hoechst solution).

Imaging was taken with a Zeiss AxioImager Z1 fluorescence microscope, supplemented with Apotome semiconfocal setup, 400× magnification, the same exposition time.

Examining T544-1567 (inhibitor), hsFlp; pAct<CD2<Gal4, UAS-nlsGFP, r4:mCherry:Atg8a flies were crossed with animals of yw; (EP)EDTP^{EY22967} genotype (the autophagy antagonist EDTP/Jumpy is clonally overexpressed in fat body).

Cells with overactivated EDTP/Jumpy are in green due to the presence of the GFP transgene. Blue colour (DAP staining) indicates the nuclei (DNA). Red dots (mCherry:Atg8) indicate autophagy structures (autophagosomes+autolysosomes). The fat body was prepared form control (non-treated) and treated flies—this tissue consists of large, polyploid cells, in which stress-induced autophagy can relatively easily be visualized.

### Results:

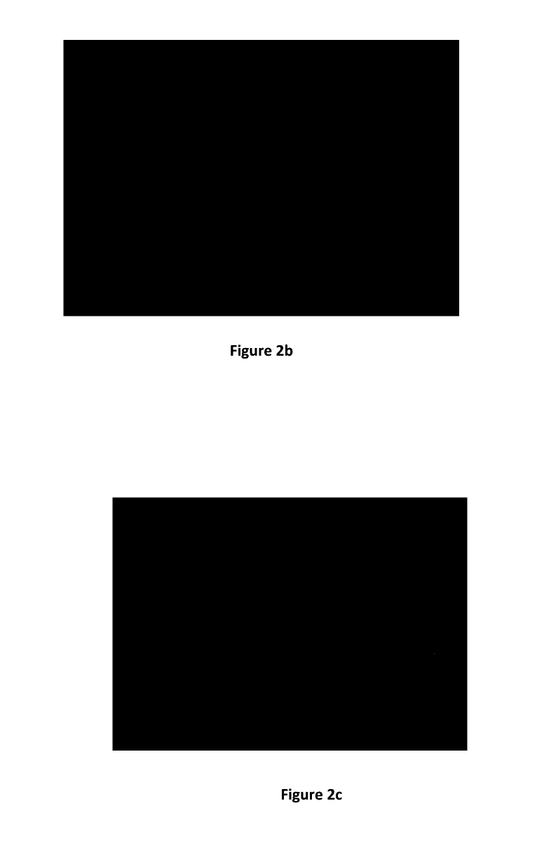

Blue colour (DAP staining) indicates the nuclei (DNA). Red dots (mCherry:Atg8) indicate autophagy structures (autophagosomes and autolysosomes). The image depicted in FIG. 2a was taken on negative control—non treated—larvae. Only a very few red foci are visible, representing a basal level of autophagy in a well-fed larva.

FIG. 2b shows a larva treated with 1.0 uM T558-0696. Blue colour (DAP staining) indicates the nuclei (DNA). Red dots (mCherry:Atg8) indicate autophagy structures (autophagosomes+autolysosomes). An increased number of red dots are visible, representing an increased autophagic activity in response to the higher dose (100 uM) treatment.

### 3) The Effects of Autophagy Inducing Compounds in Drosophila on Life-Span Essays

### Methods

For lifespan assays, flies with the w¹¹¹⁸ (wild-type) genotype were used. 10 μM, 20 μM and 30 μM of autophagy stimulating compounds were added to yeast suspensions, from which 100 ul were taken and dried on the surface of normal solid media. Each test contained 100 male and 100 female flies (animals were cultured in glass tubes—20-20 males and females/tube). Animals were transferred to new tubes with freshly prepared agents in every 2-days, and dead animals were counted by 2 days. Flies were maintained at 25 degree. Active substances were dissolved in DMSO (controls also contained DMSO). Statistics were performed by SPSS software, Kaplan-Meyer curves were generated for survival data.

### Results

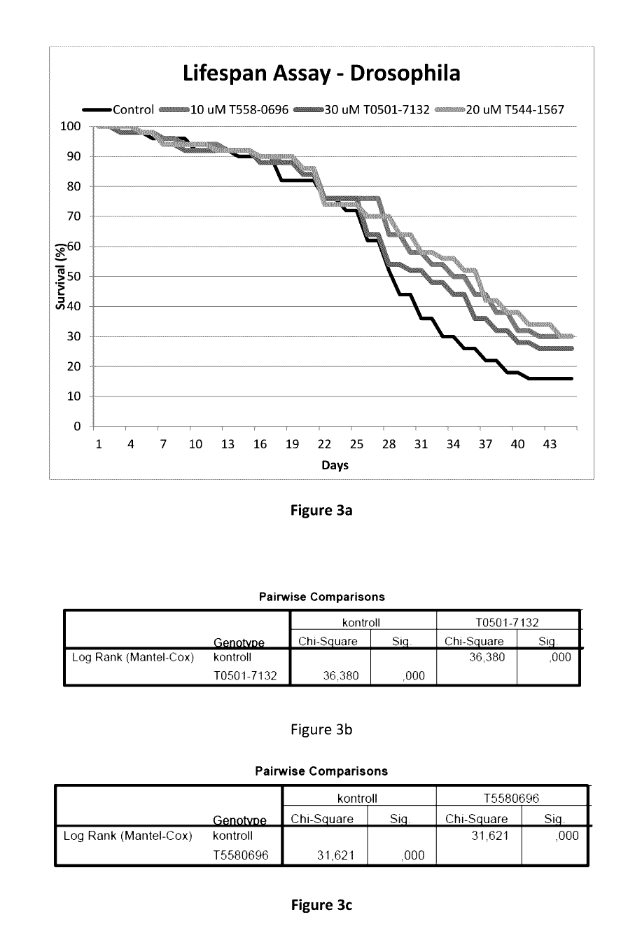

The autophagy inducing compounds extend the lifespan in Drosophila, and this longevity effect is particularly evident at advances ages. (FIG. 3a) The differences are statistically highly significant (Mantel-Cox longrank test, p<0.0001) (FIGS. 3b-c).

### 4) The Effects of Autophagy Inducing Compounds on Autophagic Activity in Mice

### Methods

50 uMol of effective molecules were dissolved in 10% DMSO and were administered intraperitoneously. After 2.5 hours the animals were sacrificed and their organs removed. The organs were transferred to tissue cultivating solution.

The organs were prepared for electronmicroscopic studies by standard preparation techniques. The ultrathin slices were prepared using Reichert-Jung Ultracut-E type ultramicrotome. The slides were investigated by JEM Jeol 1011 electron microscope.

### Results:

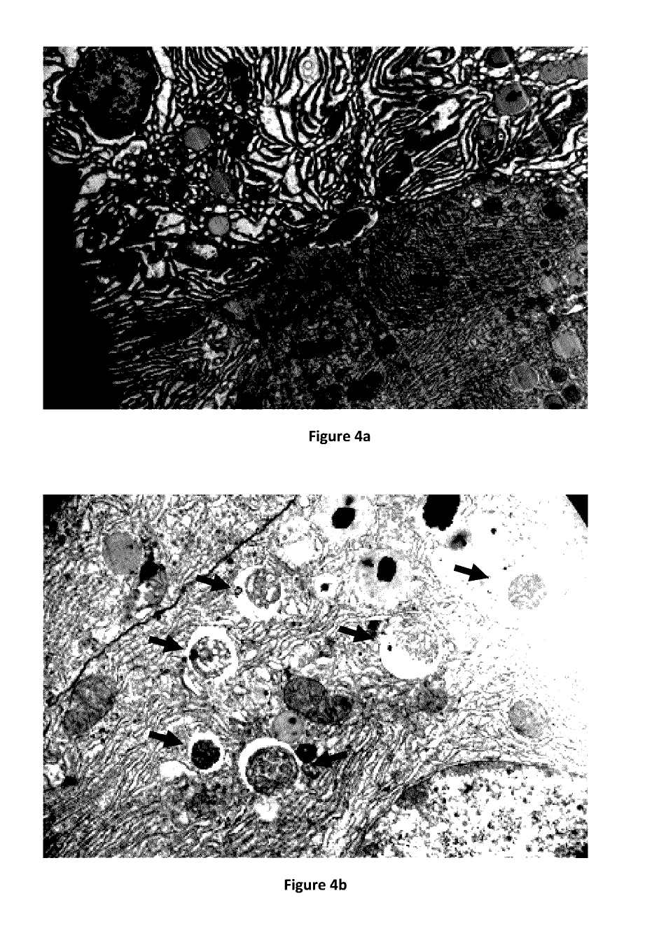

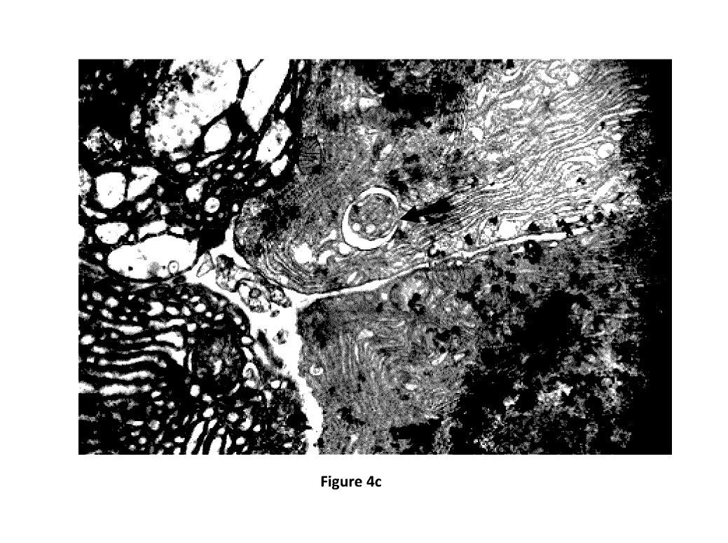

FIG. 4a shows that if treated with control (100 μl DMSO), no autophagic structures are visible in the pancreas.

The administration of 50 uMol T558-0696 shows pronounced autophagic activity represented by autophagosomes and autolysosomes (indicated by arrows) in pancreas (FIG. 4b) and in liver of mice (FIG. 4c)

While this invention has been particularly shown and described with references to preferred embodiments thereof, it will be understood by those skilled in the art that various changes in form and details may be made therein without departing from the scope of the invention encompassed by the appended claims.

1. A method of promoting autophagy in a subject, comprising administering to the subject a therapeutically effective amount of autophagy inducing compounds of Formula I, II or II, or a pharmaceutically acceptable salt, hydrate, tautomer or prodrug thereof: 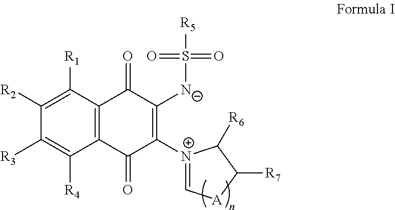
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 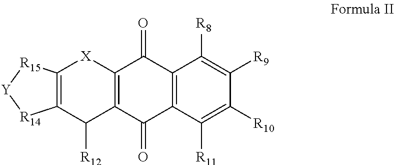
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl halo-, nitro-, hydroxyl, amino, and alkoxy;
Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy; 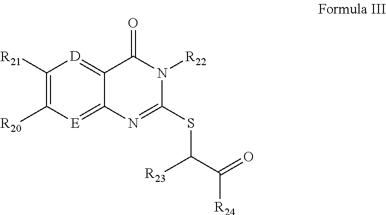
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

2. A method of treating an autophagy related disorder comprising administering to a subject in need thereof, a therapeutically effective amount of autophagy inducing compounds of formula I, II or III, or a pharmaceutically acceptable salts, hydrate, tautomer or prodrug thereof, 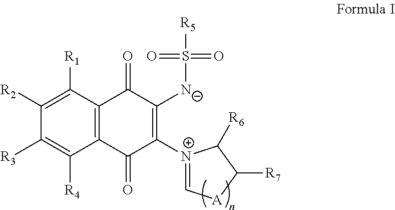
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 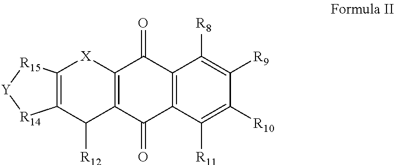
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl, halo-, nitro-, hydroxyl, amino, and alkoxy;
Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy; 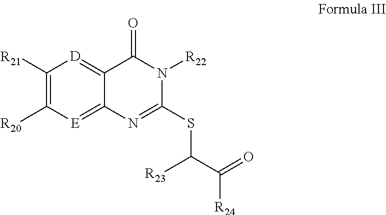
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

3. A method of claim 2 wherein said autophagy related disorder is selected from cancer, stroke, sarcopenia, infection, liver disease, neurodegenerative disease and cardiac disorders.

4. A method of claim 3 wherein said neurodegenerative disease is selected from Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjögren-Batten disease), Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), MELAS—Mitochondrial Encephalopathy, Lactic Acidosis and Stroke, Multiple System Atrophy, Multiple sclerosis, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Spinocerebellar ataxia (multiple types with varying characteristics), Spinal muscular atrophy, Steele-Richardson-Olszewski disease, Tabes dorsalis, Tay-Sachs Disease, and Toxic encephalopathy.

5. The method of claim 3, wherein said cancer is bone cancer, colon cancer, multiple myeloma, gastric cancer, colorectal cancer, prostate cancer, cervical cancer, lung cancer, pancreatic cancer, medulloblastoma, liver cancer, parathyroid cancer, endometrial cancer or breast cancer.

6. A method of promoting longevity in a subject, comprising administering to the subject a therapeutically effective amount of autophagy inducing compounds of Formula I, II or III, or a pharmaceutically acceptable salt, hydrate, tautomer or prodrug thereof 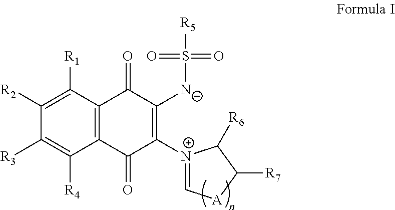
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 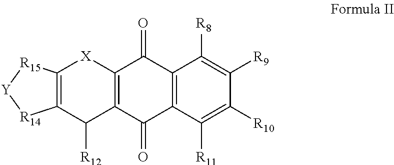
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl, halo-, nitro-, hydroxyl, amino, and alkoxy;
Y is C(O) or CHR¹⁹, where R⁹ is selected from hydrogen, branched or linear aliphatic, alicyclic, aryl, heterocyclic, heteroaryl, halo-, nitro-, hydroxyl, amino, cyano, and alkoxy; 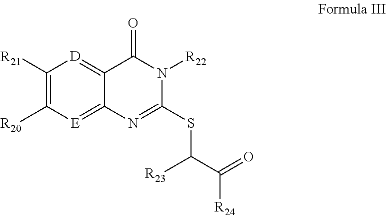
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

7. A method of alleviating or preventing premature aging in a subject, comprising administering to the subject a therapeutically effective amount of autophagy inducing compound of Formula I, II or III, or a pharmaceutically acceptable salt, hydrate, tautomer or prodrug thereof: 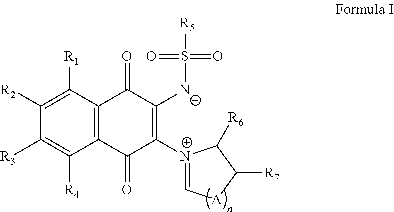
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 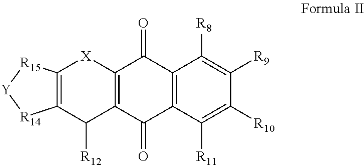
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl, halo-, nitro-, hydroxyl, amino, and alkoxy;
Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy; 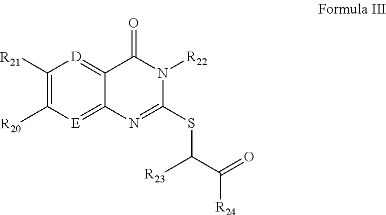
Wherein
R²⁰ and R²¹ are each independently selected from hydrogen, branched or linear aliphatic, alicyclic, aryl, heterocyclic, heteroaryl halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

8. An autophagy inducing compound of Formula I, II or II, or pharmaceutically acceptable salt, hydrate, tautomer or prodrug thereof for use in a method of promoting autophagy: 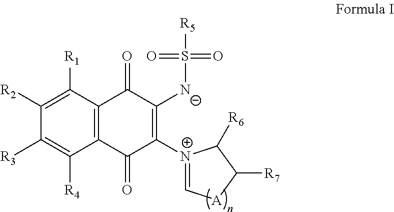
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 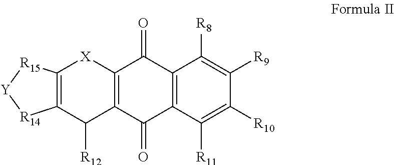
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where V hydrogen, branched or linear aliphatic, alicyclic, aryl, heterocyclic, heteroaryl, halo-, nitro-, hydroxyl, amino, and alkoxy;
Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy; 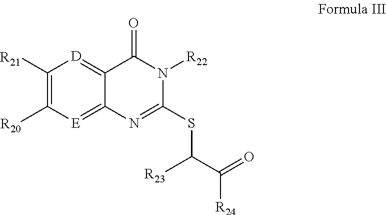
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

9. An autophagy inducing compound of Formula I, II or III, or a pharmaceutically acceptable salt, hydrate, tautomer or prodrug thereof for use in a method of treating an autophagy related disorder 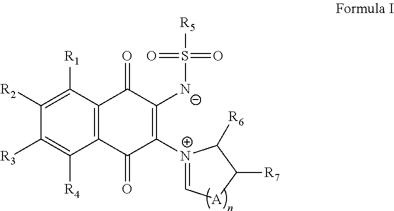
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 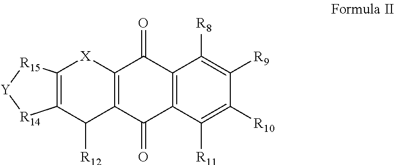
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl, halo-, nitro-, hydroxyl, amino, and alkoxy;
Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy; 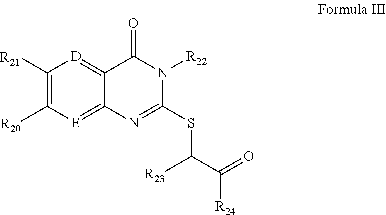
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

10. The autophagy inducing compounds for use of claim 9 wherein said autophagy related disorder is selected from cancer, stroke, sarcopenia, infection, neurodegenerative disease and cardiac disorders.

11. The autophagy inducing compounds for use of claim 10 wherein said neurodegenerative disease is selected from Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjögren-Batten disease), Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), MELAS—Mitochondrial Encephalopathy, Lactic Acidosis and Stroke, Multiple System Atrophy, Multiple sclerosis, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Spinocerebellar ataxia (multiple types with varying characteristics), Spinal muscular atrophy, Steele-Richardson-Olszewski disease, Tabes dorsalis, Tay-Sachs Disease, and Toxic encephalopathy.

12. The autophagy inducing compounds for use of claim 10 wherein said cancer is bone cancer, colon cancer, multiple myeloma, gastric cancer, colorectal cancer, prostate cancer, cervical cancer, lung cancer, pancreatic cancer, medulloblastoma, liver cancer, parathyroid cancer, endometrial cancer or breast cancer.

13. An autophagy inducing compound of Formula I, II or III, or a pharmaceutically acceptable salt, hydrate, tautomer or prodrug thereof for use in a method of increasing longevity 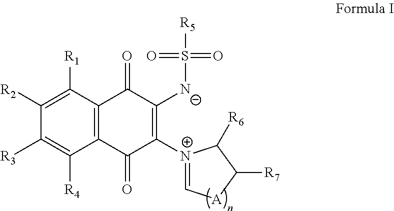
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 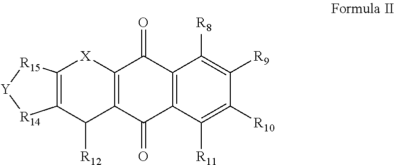
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl, halo-, nitro-, hydroxyl, amino, and alkoxy;
Y is C(O) or CHR¹⁹, where R¹⁹ is selected from hydrogen, branched or linear aliphatic, alicyclic, aryl, heterocyclic, heteroaryl, halo-, nitro-, hydroxyl, amino, cyano, and alkoxy; 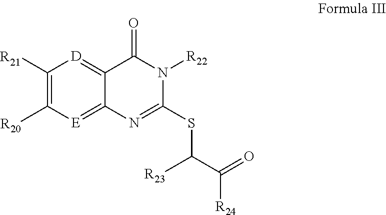
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

14. An autophagy inducing compound of formula I, II or II, or pharmaceutically acceptable salt, hydrate, tautomer, or prodrug thereof for use in a method of alleviating or preventing premature aging Formula I: 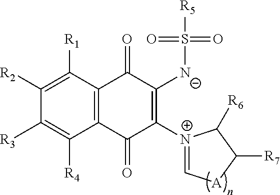
Where R¹, R², R³, R⁴ and R⁵ are each independently selected from hydrogen, branched or linear aliphatic, alicyclic, aryl, heterocyclic, heteroaryl, halo-, nitro-, hydroxyl, amino, cyano and alkoxy;
A is selected from C(R^{a})ₘ or NR^{a} wherein are each R^{a} is independently selected from hydrogen, branched or linear aliphatic, alicyclic, aryl, heterocyclic, heteroaryl, halo-, nitro-, hydroxyl, amino, and alkoxy;
n is 1 or 2;
m is 1 or 2; when m is 1 the bond between the carbon atoms attached to R⁶ and R⁷ is a single bond
R⁶ and R⁷ are each independently selected from hydrogen, branched or linear aliphatic, alicyclic, aryl, heterocyclic, heteroaryl, halo-, nitro-, hydroxyl, amino, cyano and alkoxy; or R⁶ and R⁷ together form an optionally substituted 5 or 6 membered alicyclic or heterocyclic aryly or heteroaryl ring; 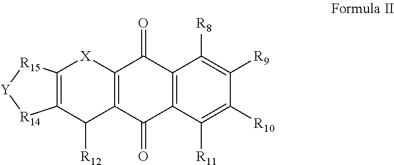
Where R⁸, R⁹, R¹⁰, and R¹¹ are each independently selected from hydrogen, branched or linear aliphatic, alicyclic, aryl, heterocyclic, heteroaryl, halo-, nitro-, hydroxyl, amino, cyano, and alkoxy;
R¹² is selected from aryl group, or a heterocyclic group, optionally substituted with R¹³, where R¹³ is selected from hydrogen, branched or linear aliphatic, alicyclic, aryl, heterocyclic, heteroaryl, halo-, nitro-, hydroxyl, amino, cyano and alkoxy;
R¹⁴ and R¹⁵ are each independently CHR¹⁶ or a heteroatom selected from sulphur, oxygen or N—R¹⁷, where R¹⁶ and R¹⁷ are each independently hydrogen, branched or linear aliphatic, alicyclic, aryl, heterocyclic, heteroaryl, halo-, nitro-, hydroxyl, amino, cyano, and alkoxy;
X is a heteroatom selected from sulphur, oxygen or N—R¹⁸, where R¹⁸ hydrogen, branched or linear aliphatic, alicyclic, aryl, heterocyclic, heteroaryl, halo-, nitro-, hydroxyl, amino, and alkoxy;
Y is C(O) or CHR¹⁹, where R⁹ is selected from hydrogen, branched or linear aliphatic, alicyclic, aryl, heterocyclic, heteroaryl, halo-, nitro-, hydroxyl, amino, cyano, and alkoxy; 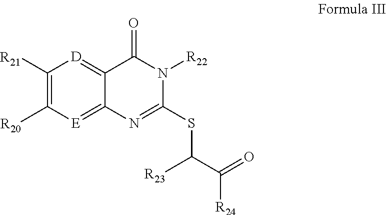
Wherein
R²⁰ and R²¹ are each independently selected from hydrogen, branched or linear aliphatic, alicyclic, aryl, heterocyclic, heteroaryl, halo-, nitro-, hydroxyl, amino, cyano, and alkoxy;
R²² is selected from hydrogen, branched or linear aliphatic, alicyclic, aryl, heterocyclic, heteroaryl, halo-, nitro-, hydroxyl, amino, cyano, and alkoxy;
R²³ selected from hydrogen, branched or linear aliphatic, alicyclic, aryl, heterocyclic, heteroaryl, halo-, nitro-, hydroxyl, amino, and alkoxy group;
R²⁴ is selected from alicyclic, aryl, heteroaryl or heterocyclic group;
D and E are each independently CH—R²⁵ where R²⁵ is independently selected from hydrogen, branched or linear aliphatic, alicyclic, aryl, heteroaryl, heterocyclic, halo-, nitro-, hydroxyl, amino, cyano, and alkoxy.

15. The method of claim 1 wherein R¹, R², R³, and R⁴ are each independently selected from H, C₁₋₆ alkyl or halo-.

16. The method of claim 1 or the compound for use of wherein R⁵ is an optionally substituted aryl group.

17. The method of claim 1 or the compound for use of wherein n is 1 and A is NR^{a}.

18. The method of claim 1 or the compound for use of wherein R⁶ and R⁷ together form an optionally substituted 6 membered heterocyclic ring.

19. The method of claim 1 or the compound for use of wherein R⁸, R⁹, R¹⁰, and R¹¹ are each independently selected from H, C₁₋₆ alkyl or halo-.

20. The method of claim 1 or the compound for use of wherein R¹² is an optionally substituted aryl group.

21. The method of claim 1 or the compound for use of wherein Y is C(O).

22. The method of claim 1 or the compound for use of wherein X is S.

23. The method of claim 1 or the compound for use of wherein R¹⁴ is a heteroatom.

24. The method of claim 1 or the compound for use of wherein R¹⁵ is N—R¹⁷.

25. The method of claim 1 or the compound for use of wherein R²⁰ and R²¹ are each independently selected from H, C₁₋₆ alkyl or halo-.

26. The method of claim 1 or the compound for use of wherein R²² is selected from hydrogen, branched or linear aliphatic, alicyclic, aryl, heteroaryl and heterocyclic groups.

27. The method of claim 1 or the compound for use of wherein R²⁴ is an optionally substituted aryl or heteroaryl group.

28. The method of claim 1 or the compound for use of wherein R²⁵ is selected from H, C₁₋₆ alkyl or halo-.

29. The method of claim 1 or the compound for use of wherein the compound is: 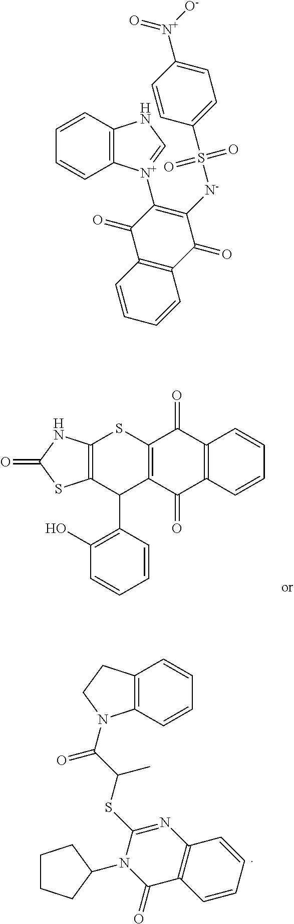 [SMILES: O=C1C([N-]S(=O)(=O)c2ccc([N+](=O)[O-])cc2)=C([n+]2c[nH]c3ccccc32)C(=O)c2ccccc21 ; O=C1C2=C(C(=O)c3ccccc31)C(c1ccccc1O)c1sc(=O)[nH]c1S2 ; CC(Sc1nc2ccccc2c(=O)n1C1CCCC1)C(=O)N1CCc2ccccc21]
